(19) United States
(12) Patent Application Publication
(10) Pub. No.: US 2007/0041908 A1
(43) Pub. Date: Feb. 22, 2007

(54) Injectable composite for the magnetocytolysis of bone metastatic cells

(76) Inventors: Patrick Pierre Frayssinet, Saiguede (FR); Nicole Francine Rouquet, Toulouse (FR)

Correspondence Address: FOLEY AND LARDNER LLP;SUITE 500, 3000 K STREET NW, WASHINGTON, DC, 20007, US

(21) Appl. No.: 10/563,603

(22) PCT Filed: Jul. 7, 2004
(86) PCT No.: PCT/FR04/01766

§ 371 Date: Jun. 28, 2006

(30) Foreign Application Priority Data
Jul. 8, 2003 (FR) 03/08332

(51) Int. Cl. A61K 49/10; A61K 9/14; A61K 33/26

(52) U.S. Cl. 424/9.34; 424/484; 977/906; 424/646

(57) ABSTRACT

The invention relates to a degradable, biocompatible material which is made from a phosphocalcic cement comprising magnetic particles. Said material can be used to treat bone metastases by means of thermolysis and/or to mark cancer cells.

The present invention relates to a degradable biocompatible material consisting of a phosphocalcium matrix comprising magnetic particles, said material being useful for treating bone metastases by thermolysis and/or for tracing cancer cells.

Bone metastases of primary cancers located elsewhere in the organism are very frequent; they are even an expected development of the disease, as the patients' life expectancies increase. Bone tissue is one of the most frequent localizations of cancer metastases. They are particularly frequent in the natural history of breast, prostate, lung, kidney and thyroid cancers. Radiologically in the majority of the cases, they form a lacuna because of the high osteolytic activity which occurs in their periphery. This osteolysis is clinically accompanied by pains in the bone and by fractures of long bones or compressions of vertebral bodies. Prognosis of bone metastases remains very poor and their treatment is palliative. Extended survivals may however be obtained depending on the characteristics of the primary tumor.

Histologically, metastases are classified into osteolytic tumors, mixed osteoblastic tumors and tumors of the intertrabecular type. The most frequent type is the mixed type in which a bone resorption area coexists with a regeneration area at the periphery of the former. Metastases consist of cell clusters between trabeculae or remains of trabeculae. At a cell level, there is great polymorphism and many atypical cells with many mitotic images. They are surrounded with bone tissue showing many signs of resorption as numerous Howship's lacunae with active osteoclasts.

The cells forming these cell clusters stem from the migration of cells from the primary tumor, which fix themselves in the bone tissue as they find favorable conditions for their growth. Bone metastases may also be classified into different stages of development: appearance phase, interaction phase, and carcinomatous phase. Most often, they are diagnosed during the interaction phase, i.e., when the tumor cells activate formation and activity of osteoclasts which are the cells responsible for bone resorption. It seems that tumor cells collaborate with stromal cells and osteoblasts for recruiting osteoclasts through the RANK-RANKL system (Kitazawa, S. and Kitazawa, R., RANK ligand is a prerequisite for cancer-associated osteolytic lesions, J. Pathol., 2002: 198; 228-236).

The object of the invention is the treatment of bone metastases. It allows cancerous cells to be removed in order to limit progression of the disease and to abolish stimulation of the osteoclasts without, in as far as possible, destroying bone regeneration capacities at the periphery of the tumor. Removal of cancer cells clinically results in reduction of the pain and lowering of the risk of fracture.

Several of treatments are usually associated so as to reduce the tumor volume. External radiotherapy is common, often associated with chemotherapeutical treatment. Evading. chemotherapy is relatively early and hematological risks as well as discomfort inherent to chemotherapy by a general route remain a major problem of this type of treatment. Radiotherapy is not devoid of risks, in particular, when the metastasis is located near a noble organ, lung, central nervous system. On the other hand, the problem of these treatments, when they are effective, stem from concomitant removal of the cells involved in bone regeneration.

Other active treatments at the metastasis may be applied with an antalgic purpose. Intravenous injection of a strontium isotope as well as the use of biphosphonates was proved to be effective. These compounds do not lead or only very slightly to removal of cancer cells.

Original treatment routes are presently being developed so as to have a more specific action on the cancerous cell. They are intended to avoid secondary effects of chemotherapy and radiotherapy which cause cytolysis of rapidly proliferating cells and are therefore toxic to blood stem cells and those of epithelia with fast renewal. They are based on the principle of transporting an inactive molecule into contact with tumor cells, the molecule being activated when it has reached the tumor. Fixing cytotoxic molecules on magnetic particles which will be injected intravenously and which are concentrated in the tumor by means of a magnetic field or specific organic molecules of the receptors of tumor cells is a method which has given rise to many studies (Yanase, M., Shinkai, M., Honda, H., Wakabayashi, T., Yoshida, J., Kobayashi, T., Intracellular hyperthermia for cancer using magnetite cationic liposomes: an in vivo study, Jpn. J. Cancer. Res.; 89 (4): 463-9, 1998; Moroz, P., Jones, S., Gray, B. N., Magnetically mediated hyperthermia: current status and future directions, Int. J. Hyperthermia; 18: 267-84, 2002; Pulfer, S. K., Gallo, J. M., Targeting magnetic microsphere to brain tumors, in: Hafeli, U., Schütt, W., Teller, J., Zborowski, M., (eds.) Scientific and clinical applications of magnetic carriers. Plenum Press New York 1997 pp 445-456. The particles once phagocytized or internalized by cells may have specific toxicity by the active ingredient which they carry and which they again expel into the cell. They may also be heated in a high frequency magnetic field and they may induce direct thermolysis or else indirectly by sensitizing heated cells to radiotherapy or chemotherapy.

The major obstacles to this type of therapy stem from the more or less correct specificity for addressing the particles. Depending on their surface features and plasma proteins being fixed on their surface, they will either be eliminated or not by phagocytosis in the spleen and the lever by macrophages before they are activated at the contact of cancer cells (Müller, R. H., Luck, M., Harnisch, S., Thode, K., Intravenously injected particles: surface properties and interaction with blood proteins—The key determining the organ distribution, in: Hafeli, U., Schütt, W., Teller, J., Zborowski, M., (eds) Scientific and clinical applications of magnetic carriers. Plenum Press New York 1997, pp 135-148). Another obstacle is related to the more or less large specificity of the molecules responsible for cellular recognition as well as the possibility, for the particles to pass through the vascular barrier.

The possibility of directly injecting into the tumor, micro particles suspended in a liquid as also been mentioned. However, injection is delicate as it is difficult under these conditions to know where the particles are going. The particles should be able to penetrate into the tumors and then into the cells which they should destroy. Effectiveness of the particles in thermocytolysis is related to their contact with the cells. Particles which are not bound to the cells are much less effective (Bacri, J. C., de Fatima Da Silva, M., Perzynski, R., Pons, J. N., Roger, J., Salbolovic, D., Halbreich, A., Use of magnetic nanoparticles for thermolysis of cells in a ferrofluid. in: Hafeli, U., Schütt, W., Teller, J., Zborowski, M., (eds) Scientific and clinical applications of magnetic carriers. Plenum Press New York 1997, pp 597-606). The cells of the mononucleated phagocyte system present in the blood and of numerous tissues are capable of phagocytosing particles up to a size of about forty μm. The other cells have much more limited phagocytosis capacities. On the other hand, nearly all the cells are capable of ingesting particles with a much reduced size by endocytosis. Generally, it is considered that particles with a size less than 100 nm are capable of passing through the cell membrane by endocytosis. For this, it is sufficient that the cells be in contact with the particles.

Thus, finding alternative therapeutical solutions appears to be necessary in order to meet the aforementioned problems concerning the use of magnetic particles in cancerology.

Within the scope of the invention, we have developed a system with which a slurry containing a mineral suspension may be injected into a tumor from which calcium sulfate or phosphate will precipitate, containing magnetic particles with small sizes dispersed in the formed mineral matrix which will be released during degradation of the latter. The role of the matrix is to confine the particles in the injection area, to keep them separate from each other and to release them at the contact of tumor cells according to defined kinetics. The released particles will then penetrate into the tumor cells by endocytosis.

Several advantages are obtained with this material: matrices of calcium phosphates and sulfates have good bone biocompatibility. For the most part they are capable of being totally integrated into the bone tissue without causing any significant reaction to a foreign body. They are then degraded at variable rates according to their chemical composition and their physico-chemical characteristics and totally replaced by bone tissue. These matrices are injected into the bone as a slurry from which a calcium phosphate sulfate compound precipitates, different from the one which is suspended in the slurry. Entanglement of the precipitate's crystals provides setting of the material and its mechanical strength which may be close to that of spongious bone within a few hours to a few days (T. Yuasa, Y. Miyamato, K. Ishikawa, M. Takechi, M. Nagayama, and K. Suzuki. In vitro resorption of three apatite cements with osteoclasts. J. Biomed. Mater. Res. 54:344-353, 2001; S. Takagi, L. C. Chow, M. Markovic, C. D. Friedman, and P. D. Costantino. Morphological and phase characterizations of retrieved calcium phosphate cement implants. J. Biomed Mater Res (Appl Biomater) 58:36-41, 2001; Y. Miyamato, T. Toh, T. Yuasa, M. Takechi, Y. Momota, M. Nagayama, K. Ishikawa, and K. Suzuki. Basic properties of apatite cement containing carbonate apatite and its resorption by cultured osteoclasts. In: Proceedings of the 13^{th} Int. Symp. on Ceramics in Medicine, A Anonymous Switzerland: 2001, p. 829-832).

Moreover, magnetic particles may be heated in an electromagnetic field before or after their having penetrated into the cells. This heating may be repeated the number of times required without requiring any other injection. The magnetic particles are actually released over several days to several weeks from the mineral matrix. When the cells are thermolysed, the particles which they contain are added to the ones which have just been released from the matrix so as to be phagocytized by new cells. Further, these particles acting at the core of the tumor do not interfere with bone regeneration existing in the periphery of the bone metastasis.

### DESCRIPTION

Thus, the invention relates to a degradable biocompatible material characterized in that it consists of a degradable biocompatible phosphocalcium and/or calcium sulphate matrix or of a degradable biocompatible polymer matrix, said matrix containing magnetic particles, said material being found as a slurry during its introduction into the organism and as a solid subsequently. The particles are released during degradation of the cement forming the matrix in which they are trapped.

By “phosphocalcium matrix”, a mixture is meant, comprising one or more selected phosphates from the group of amorphous calcium phosphates, low-crystalline apatite phosphates, anhydrous dicalcium phosphates or dicalcium phosphate dihydrates, tricalcium phosphates, monocalcium phosphate monohydrates, pyrophosphates, octocalcium phosphates, or hydroxyapatite. Preferably, the phosphocalcium matrix is rapidly resorbable, i.e., within a period of a few days to a few weeks which implies a solubility corresponding to that of dicalcium phosphate. Said matrix may also consist of all or part of calcium sulfates which have biocompatibility and degradation characteristics also compatible with the applications of the material to the treatment of bone tumors. Such a material according to the invention is a mineral material introduced as a slurry. During setting, precipitation of a phase does not occur in the suspension or else it is a minority in the latter. The first histological resorption signs (notches, partial fragmentation) are visible under the microscope a few days after its introduction, and radiological disappearance occurs between 4-52 weeks following composition of the mineral phase, preferentially 8 weeks for calcium sulfate matrices.

In addition, said material may consist of a degradable polymer matrix containing magnetic particles. It may consist of a natural or artificial biodegradable polymer, such as collagen, polylactic and glycolic acids, polydioxanone, polyfumarate, polyanhydrides, polyorthoesters, polyurethanes, polyphosphazenes, polycaprolactone, polyhydroxy-butyrate, polyhydroxyvalerate, polyvalerolactone, polytartronic and polymalonic acid. The material may be in the form of a gel, foam or slurry.

By “magnetic particles”, are meant particles containing a metal, notably iron, preferably as ferrites: magnetite or maghemite or any other ferro-, ferri-magnetic, meta- or anti-ferromagnetic inorganic material. They preferably consist of ferrite (Fe₂O₃) or magnetite. They may also exist as an organomineral composite. The magnetic mineral forming the core of the particle is surrounded with a layer of an organic compound. The metal particles may be obtained by hydrothermal synthesis in a stirred reactor by injecting 80 ml of a FeCl₂ solution, calculated so as to be able to synthesize 5 g of magnetite, 170 m³ of deaerated water containing 10 g of NaOH are then added. Under nitrogen flow (30 l/hr) the solution is brought to 80° C. When this temperature is reached, nitrogen is replaced with compressed air at the same flow rate for 20 hours. The ferrites are then washed with water and then ethanol before being dried.

Preferably, the magnetic particles have a particle size between 0.001 μm and 0.01 μm, or further between 0.05 μm and 0.1 μm, for example 0.07 μm, 0.15 μm, 0.5 μm. However, they may have a size of the order of 1 micron for particular applications, for example a particle size between 0.1 and 10 μm.

Such a material thus forms a mineral matrix releasing the magnetic particles according to kinetics compatible with their internalization by the cells of neighboring tissues. The material of the invention may reside in an association with a mineral or organic matrix of magnetic particles or iron particles coated with a mineral layer, preferably calcium phosphate, sulfate or carbonate mineral. This coating may also contain a fluorescent element such as europium. More specifically, said particles consist of an organomineral composite containing an iron, ferrite core or any other magnetic compound coated with a polymer as a thin layer or as polymer chains having a free end. Advantageously, said magnetic particles are vectors of a molecule used in chemotherapy or else an isotope.

In a second aspect, the invention deals with a method for preparing said material comprising mixing of a powder of magnetic particles with a calcium sulfate or phosphate mineral powder in an aqueous solution until a slurry is formed, and hardening said slurry for a few minutes to a few hours. This method may further comprise a step for preparing said particles by hydrothermal synthesis in a reactor, by injecting a solution of FeCl₂, adding deaerated water containing NaOH, the mixture being placed under nitrogen flow and brought to a temperature between 50° C. and 100° C., replacing nitrogen with compressed air until ferrites are obtained.

The magnetic particles once inside the cells are intended to be heated in a magnetic field which may for example be produced by a nuclear magnetic resonance imaging apparatus or by any other generator. Thus, in a third aspect, the invention relates to the use of a material described above for preparing a drug or a medical device for treating bone tumors. More particularly, this medical device provides targeted thermolysis of cancer cells. This is made possible by means of the magnetic particles which are capable of inducing hyperthermia in the tissues in which they are released.

One of the advantages of this heating method in an electromagnetic field is the possibility, as soon as the injection is performed, of repeating it the number of times required without requiring another injection. The magnetic particles are actually released over several days to several weeks from the phosphocalcium matrix. When the cells are lysed, the particles which they contain are added to those which have just been released from the calcium matrix in order to be phagocytized by new cells. On the other hand, these particles acting at the core of the tumor do not interfere with bone regeneration existing in the periphery of bone metastases.

Another advantage is the possibility of combination with other presently known treatment methods, notably radiotherapy and/or chemotherapy.

In a fourth aspect, the invention deals with a method for diagnosing extension of bone cancers, comprising the use of magnetic particles as tracers of MRI-detectable tumor cells and the tracking of the migrating cells in order to be able to treat sites at infraclinic stages. It should be emphasized that for tracing cells, iron oxide particles with a larger size (˜0.5 μm) produce a signal with a stronger intensity (Hinds, K. A., et al. Highly efficient endosomal labelling of progenitor and stem cells with large magnetic particles allows magnetic resonance imaging of single cells, Blood, 2003).

The invention also relates to a method providing the tracing of cells which have ingested said particles after desalting from a degradable and biocompatible material as described above, by means of MRI, electronic microscopy, confocal microscopy, or fluorescence microscopy.

### EXAMPLE 1

### Characteristics of the Magnetic Particles

Obtained by hydrothermal synthesis
Composition Fe₂O₃
Octogonal shape
Magnetic properties: H_{c}=350 Oe, σᵣ=32 uem/g
Size 50-1,500 nm.

### EXAMPLE 2

### Characteristics of the Calcium Sulfate Powder

Composition CaSO₄.2H₂O: 96%
CaCO₃.MgCO₃: 2.1%
Fe₂O₃.Al₂O₃: 0.5%
SiO₂: 0.4%
Average particle size: 17-20 μm
pH of the 10% mixing solution: 7.6
Solubility for 100 ml of water: 0.3 g
Specific surface area: 2.5

### EXAMPLE 3

### Endocytosis of Magnetic Particles

1 mg of magnetic particles as described earlier with a particle size of 0.07 μm was introduced into a primary metastatic bone cell line culture in order to check the cells' capabilities of phagocytosing the particles. The cell line was obtained from biopsy of a bone metastasis from a breast adenocarcinoma during an osteosynthesis operation. The cells contained in the biopsy were dissociated by incubation for two hours at 37° C. in a collagenase solution in isotonic phosphate buffer. The cell suspension was then centrifuged, re-suspended in 2 ml of DMEM culture medium supplemented with 5% of calf fetal serum and introduced into a culture flask at a density of 10⁵ cells/ml. The cells were grown for three days to a pre-confluence stage. They then form a carpet leaving a gap between the cells, some of which are spherical, thereby indicating high proliferation. 1 mg of powder is then suspended with stirring in 1 ml of culture medium and 0.5 ml is introduced into the culture flask in which it is diluted by addition of 3 ml of culture medium. The cells are then incubated at 37° C. for 48 hours. After this growth period, the cells are observed in inverted optical microscopy and then the bottom of the dish is scraped and the collected cells are dehydrated, incorporated into epoxy resin. 250 angstrom cut sections are made and observed under a transmission electronic microscope. The optical microscopy examination reveals particles of different sizes (cluster of particles) inside the cytoplasm of the cells. The electronic microscopy sections show that the cytoplasm contains many lysosomal type vesicles, filled with one or more metallic particles. Between 10 and 30% of the sectional surface areas of the cells are occupied by particles.

### EXAMPLE 4

### Test of the Different Particle Sizes

1 mg of the magnetic particles described earlier was introduced into a primary line culture of the same line of metastatic bone cells in order to check the capabilities of these cells as to phagocytosing particles. The particles have four different particle sizes: 0.07 μm, 0.15 μm, 0.5 μm. The cell line was obtained from biopsy of a bone metastasis of a breast adenocarcinoma. The cells contained in the biopsy were dissociated by incubation for two hours at 37° C. in a collagen solution in an isotonic phosphate buffer. The cell suspension was then centrifuged, re-suspended in 2 ml of DMEM culture medium supplemented with 5% calf fetal serum and introduced into a culture flask at a density of 10⁵ cells/ml. The cells were grown for three days to a pre-confluence stage. They then form a carpet leaving a gap between the cells, some of which are spherical, thereby indicating high proliferation. 1 mg of powder of each sample is then suspended, with stirring, in 1 ml of culture medium and 0.5 ml is introduced into a culture flask in which it is diluted by adding 3 ml of culture medium. Each test is repeated three times. The cells are incubated at 37° C. for 48 hours. The cultures are then examined in inverted optical microscopy and in transmission electronic microscopy.

Regardless of the particle size, within a few hours, many particles are in contact with the cells and they have penetrated into the cells at the end of the period of observation. As reported by Hinds (Hinds, K. A., et al. Highly efficient endosomal labelling of progenitor and stem cells with large magnetic particles allows magnetic resonance imaging of single cells. Blood, 2003), internalization of the particles did not seem to have induced cell death during the growing period.

### EXAMPLE 5

### In Vivo Degradation of the Mineral Matrices

Four adult sheep were anaesthetized and a hole with a diameter of 4 mm and a length of 5 mm was made in the right external condyle. The hole was then filled with slurry consisting of calcium sulfate which was left to set in situ. Two sheep were euthanized at two weeks and the two other ones at four weeks. The condyles were sampled, dehydrated in ethanol and included in polymethyl methacrylate blocks. 7 μm thick cuts were made, colored with a Giemsa solution and observed under optical microscopy. At two weeks, the injected plaster is still visible. It is in the process of being resorbed, with notches in the mineral matrix. The implant is surrounded with loose connective tissue with monocytes and giant cells in the immediate periphery of the implant, there are signs of osteogenesis at the bone trabeculae in which the plaster is implanted. Fragments of materials are visible, and the cells in contact with the material according to the invention, contain mineral grains. At four weeks, the implant has disappeared. There remains in the implantation area, a loose connective tissue area which is much smaller than the section of the implant. Porous immature bone tissue has penetrated into the implantation area. There are still a few fragments of implants and the macrophage cells still contain a few grains of material. There is no sign of osteolysis.

### EXAMPLE 6

### In Vivo Deliverance of Magnetic Particles to the Cells By the Matrix

Four adult sheep were anaesthetized and a hole with a diameter of 4 mm and a length of 5 mm of the right external condyle was made. The hole was then filled with slurry consisting of plaster of Paris containing 2 mg of magnetic particles per ml of mixing solution. Two sheep were euthanized at two weeks and the two other ones at four weeks. The condyles were sampled, dehydrated in ethanol and included in polymethyl methacrylate blocks. 7 μm thick cuts were made, colored with a Giemsa solution and observed under an optical microscope. At two weeks, there is a beginning of resorption of the material which is surrounded with loose connective tissue. The macrophage cells in contact with the material contain birefringent mineral particles as well as black magnetic particles which achieve tattooing of the cytoplasm. It should be noted that connective cells who do not have the morphological features of macrophages also exhibit a tattooed cytoplasm. At one month, the implant has disappeared and a major part of the section surface occupied beforehand by the implant is invaded by bone trabeculae. The inter-trabecular space is occupied by a loose connective tissue, all the cells of which are tattooed by the magnetic particles. A transmission microscopy study confirms that numerous cells around the implant contain metal particles.

1. A biocompatible degradable composite material, characterized in that it consists of a degradable biocompatible phosphocalcium and/or calcium sulfate matrix, said matrix containing magnetic particles, said material being found as a slurry during its introduction into the organism, as a solid subsequently and said matrix being resorbed within a period of a few days to a few weeks.

2. The composite material according to claim 1, characterized in that the calcium phosphate is a mixture comprising a phosphate selected from the group of amorphous calcium phosphates, low crystalline apatite phosphates, anhydrous dicalcium phosphates or dicalcium phosphate dehydrates, tricalcium phosphates, monocalcium phosphate monohydrates, pyrophosphates, octocalcium phosphates, or hydroxyapatite.

3. The material according to claim 1, characterized in that said calcium phosphate forms a rapidly resorbable phosphocalcium matrix.

4. The material according to claim 1, further comprising calcium sulfate.

5. The material according to claim 1, characterized in that it further consists of a degradable biocompatible polymer matrix comprising a polymer selected from collagen, polylactic and glycolic acids, polydioxanone, polyfumarate, polyanhydrides, polyorthoesters, polyurethanes, polyphosphazenes, polycaprolactone, polyhydroxybutyrate, polyhydroxy-valerate, polyvalerolactone, polytartronic and polymalonic acid; containing magnetic particles.

6. The material according to claim 1, characterized in that said matrix has biocompatibility and degradation characteristics compatible with applications of the material for treating bone tumors.

7. The material according to claim 1, characterized in that the magnetic particles contain a metal, notably iron, preferably as ferrites: magnetite or maghemite or any other ferro-, ferri-magnetic, meta- or anti-ferromagnetic inorganic material.

8. The material according to claim 1, characterized in that said particles consist of an organomineral composite containing an iron, ferrite core, or core of any other magnetic compound coated with polymer as a thin layer or as polymeric chains having a free end.

9. The material according to claim 1, characterized in that said magnetic particles are vectors either of a molecule used in chemotherapy or an isotope.

10. The material according to claim 1, characterized in that said particles have a particle size between 0.001 and 0.1 μm.

11. The material according to claim 1, characterized in that said particles have a particle size between 0.1 and 10 μm.

12. The material according to claim 1, forming a mineral matrix releasing magnetic particles according to kinetics compatible with their internalization by cells from neighboring tissues.

13. The material according to claim 1, characterized in that it comprises particles coated with a calcium phosphate layer containing a fluorescent element such as europium.

14. A method for preparing a material according to claim 1, comprising mixing of a magnetic particle powder with a calcium sulfate or phosphate mineral powder, in an aqueous solution until a slurry is formed, and hardening said slurry for a few minutes to a few hours.

15. The method for preparing a material according to claim 10, further comprising a step for preparing said particles by hydrothermal synthesis in a reactor by injecting a FeCl2 solution, adding deaerated water containing NaOH, the mixture being placed under nitrogen flow and brought to a temperature between 50° C. and 100° C., replacing nitrogen with compressed air until ferrites are obtained.

16. A method for diagnosing bone cancers comprising administering to a subject the material of claim 1 as a tracer for MRI-detectable tumor cells and tracking migrating tumor cells that take up the tracer in order to be able to treat sites at infraclinic stages.

17. A method for tracing tumor cells in a subject having ingested the material of claim 1 after desalting from said degradable and biocompatible material by means of MRI, electronic microscopy, confocal microscopy, or fluorescence microscopy.

18. A method according to claim 16 wherein the treatment is for treating bone tumors.

19. A method according to claim 18 wherein the treatment is for targeted thermolysis of cancer cells.

20. A method according to claim 19, characterized in that the magnetic particles once inside the cells are intended to be heated in a magnetic field which may be produced by a nuclear magnetic resonance imaging apparatus or any other generator.

21. A method according to claim 17, wherein the treatment is combined with radiotherapy and/or chemotherapy.
